(19) United States
(12) Patent Application Publication
(10) Pub. No.: US 2012/0318750 A1
(43) Pub. Date: Dec. 20, 2012

(54) METHODS AND COMPOSITIONS FOR COMPLEX BINDING OF METAL IONS

(75) Inventors: Ida Helena Högberg, Sundsvall (SE); Nils Frederik Andersson, Sundsvall (SE); Kjell Håkan Edlund, Sundsvall (SE); Sten Erik Hedenström, Stode (SE); Hans Magnus Norgren, Matfors (SE)

(73) Assignee: ChemseQ International AB, Sundsvall (SE)

(21) Appl. No.: 13/512,801

(22) PCT Filed: Dec. 10, 2010
(86) PCT No.: PCT/EP10/69394

§ 371 Date: Sep. 5, 2012

(30) Foreign Application Priority Data
Dec. 11, 2009 (SE) 0950954-8

(51) Int. Cl. C02F 1/58; C07C 229/26; C07C 229/24

(52) U.S. Cl. 210/749; 554/110; 554/107; 562/565

(57) ABSTRACT

The present invention provides methods for decreasing amounts of metal ions in liquid materials and in porous solid materials surrounded by a liquid, by utilization of sequestering agents that form complexes with said metal ions as well as methods for removing and optionally recovering said metal ions from the complexes. Further, there are provided novel sequestering agents and compositions comprising sequestering agents of the present disclosure.

### TECHNICAL FIELD OF THE INVENTION

The present disclosure relates to methods for decreasing amounts of metal ions in liquid materials and in porous solid materials surrounded by a liquid, by utilization of sequestering agents that form complexes with said metal ions as well as methods for removing and optionally recovering said metal ions from the complexes. Further, there are provided novel sequestering agents and compositions comprising sequestering agents of the present disclosure.

### BACKGROUND

The presence of metal ions in water is undesired in several industrial processes. One such process is the bleaching of cellulose pulp with different types of bleaching chemicals, such as hydrogen peroxide. Metal ions, originating from the process water or from the lignocellulosic material from which the cellulose pulp has been produced, may catalyze the degradation of peroxide and thus affect the bleaching in a negative way. Thus, in bleaching of cellulose pulp, as well as in processes such as varnishing, painting, galvanizing and coating, it is desirable with a method for removing metal ions from the process water. “Further, at landfills or at places where different industrial manufacturing or mining have been performed in the past, release of metal ions such as cadmium, cobalt, chromium, mercury, manganese, copper, zinc and nickel is also undesirable, since these metals are environmentally harmful. Further, in mining and surface treatment processes metal ions often appear in rest products and liquid rest fractions. Such metal ions may be environmentally harmful and/or of significant economic interest, whereby removal and recovery would be beneficial for several reasons. Further, in personal care products, such as skin conditioners, body lotions, hair care products and hair coloring products, certain metal ions, such as copper, calcium, magnesium and iron, can be detrimental to the personal care products performance.”

A common method for sequestering metal ions in process water is with the use of specific sequestering (or chelating) agents. The most common sequestering agents include EDTA (ethylenediaminetetraacetic acid), DTPA (diethylenetriaminepentaacetic acid) and NTA (nitrilotriacetic acid). These sequestering agents form complexes (chelates) with different metal ions and these complexes normally end in some type of recipient after sequestering. The complexes are generally stored for a very long time, since the complexes as well as the sequestering agent as such (which is normally added in excess) are hardly degradable. Thus, there is a need in the art for improved methods and improved sequestering agents.

### SUMMARY OF THE INVENTION

The inventors have realized that current sequestering methods for decreasing amounts of metal ions in liquid materials and in porous solid materials surrounded by a liquid used in practice today are generally neither such that the sequestering agents used are separable nor recoverable. Therefore, an object of the present invention is to provide methods wherein sequestering agents complexed with metal ions are separable and recoverable.

To meet this object, there is provided methods for decreasing the amount of at least one metal ion in a liquid material and in porous solid materials surrounded by a liquid, comprising the steps of:

a) contacting said liquid material or porous solid material surrounded by a liquid with at least one sequestering agent such that said sequestering agent forms at least one complex with said metal ion(s);
b) removing said complex from said liquid material; and optionally
c) recovering said sequestering agent and/or said metal ion from said complex.

Moreover, there is provided sequestering agents useful in such processes. There is also is provided compositions comprising sequestering agents of the present disclosure. There is also provided novel sequestering agents of the present disclosure.

### DETAILED DESCRIPTION OF THE INVENTION

In a first aspect of the present invention, there is provided a method for decreasing the amount of at least one metal ion in a liquid material or porous solid material surrounded by a liquid, comprising the steps of:

a) contacting said liquid material or porous solid material surrounded by a liquid, with at least one sequestering agent such that said sequestering agent forms at least one complex with said metal ion(s);
b) removing said complex from said liquid material or porous solid material surrounded by a liquid; and optionally
c) recovering said sequestering agent and/or said metal ion from said complex.

In a first configuration of this aspect, said liquid material or porous solid material surrounded by a liquid is selected from an aqueous liquid, a soil, a liquid comprising sediments or sludge, a slurry and a leachate.

In the context of the present disclosure, sequestering refers to chelating, which is the formation of two or more separate bindings between a ligand and a central atom. Thus, sequestering may be a complex binding. Consequently, sequestering at least one metal ion comprising contacting said at least one metal ion with at least one sequestering agent may represent formation of two or more separate bindings between a sequestering agent and a metal atom, i.e. complex binding the sequestering agent with the metal ion. The metal ions may be metal ions in a liquid or a slurry, or in a soil. If the metal ions are in a soil, the soil may need to be pretreated so that it forms a workable liquid material before contacting the soil with the sequestering agent. Further, a leachate refers to a liquid that for instance drains from a landfill or derived from a mining process. The leachate may vary in composition depending on the age of the landfill and the type of waste that is contained in the landfill. The leachate may contain both dissolved and suspended material. Consequently, the liquid material or porous solid material surrounded by a liquid may be a liquid or a liquid comprising suspended solids, such as suspended cellulosic material. Further, the liquid material or porous solid material surrounded by a liquid may comprise different types of sediments or sludge.

In a another configuration of this aspect, said step b) comprises flotation of said complex to provide a foam on top of said liquid material, said foam comprising said complex, and removal of said foam from said liquid material.

Flotation is a separation process known to the skilled person. The flotation may for example be dissolved air flotation, induced gas flotation or froth flotation. As an example, the flotation may comprise adding a flotation agent to said liquid material. The flotation agent may for example be selected from fatty acids, resinous acids and surfactants. Further, the flotation may comprise flowing air bubbles upwards in said liquid material that has come into contact with the sequestering agent such that a foam is created on the surface of the liquid material. The flotation may also comprise the use of a propeller or rotor that initiates a flow stream upwards in said liquid material. The flotation may be performed in a flotation plant. Further, the skilled person understands, after studying the teachings of the present disclosure, how to remove the foam from said liquid material. The total volume of foam is relatively small, thus an enrichment of metal ions in the foam occurs, since the volume of foam is often less than 10% of the initial volume.

As an example, the liquid material may be a slurry of pulp fibers. The sequestering agent of the present disclosure may then be added to the pulp fibers to form complex with metal ions comprised in the slurry, and flotation of said complex may be aided by using fatty acids and resinous acids that are released from the pulp fibers as flotation agents to provide a foam comprising the sequestering agent:metal ion complex on the surface of the slurry. Removal of metal ions from pulp fibers may be performed prior to bleaching of the pulp fibers. The methods of the present disclosure further comprise recovery of both sequestering agents and metal ions from removed complexes.

Further, there is provided methods in processing the removed complex from said liquid material or porous solid material surrounded by a liquid in step b.

Thus, in another configuration of this aspect, there is provided methods of the first aspect, wherein step c) comprises

c1) precipitating said removed complex by adjusting the pH to about 0-7 to obtain an electro neutral solution comprising said complex of said at least one metal ion and said sequestering agent in precipitated form; followed by filtration of the formed precipitate.

The removed complex in c1) is typically in the form of a foam. The pH adjustment in c1) is elected dependent on the type of sequestering agent. The pH adjustment may be carried out by adding an acid, such as a mineral acid or a carbonic acid. The filtered precipitate in c1) may be stored or disposed of or may be reused in other industrial processes. In any event, the metal ions have been removed from the initial liquid material or porous solid material and the volume of the liquid material or porous solid material, initially comprising said metal ions, has been significantly reduced.

In a another configuration of this aspect, especially when said step b) comprises flotation of said complex to provide a foam on top of said liquid material, said foam comprising said complex; step c) comprises

c2) adjusting the pH of said foam to about 6-12, such as about 8-10 by addition of an electrolyte solution;
c3) applying a direct voltage current with a cathode and an anode to said electrolyte solution, whereby said at least one metal ion precipitates as a solid on said cathode by electrochemical reduction; and
c4) removal of said cathode comprising the precipitated, solid metal ions; followed by precipitating the remaining sequestering agent in the solution by adjusting the pH to about 0-7 to obtain an electro neutral solution comprising said sequestering agent in precipitated form; followed by filtration of the formed precipitate.

The adjustment of pH in c2) is typically about 8-10, such as about pH 9. The pH should not be too high since the electrochemical reduction in c3) will be ineffective. Further, the pH should not be too low since then the complex of said at least one metal ion and said sequestering agent in the foam may precipitate prior to electrochemical reduction step in c3). It is therefore relevant to keep a relatively constant pH in the process to optimize the process. The pH in step c2) may, therefore, be monitored by measurement and, if needed, adjusted by addition of an acid, such as H₂SO₄. This may reduce the concentration of hydroxide ions near the cathode and thereby optimize the metal ion precipitation on said cathode. As an alternative in c4), the remaining sequestering agent in the solution may be extracted with an organic solvent. Further, dependent on which sequestering agent is used; it may be extracted with an organic solvent such as pentane, hexane, heptane or ethers, at any appropriate stage, in order to separate it from the process.

Details of this configuration are set out in Exemplary embodiment 3.

The metal ions referred to in the present disclosure represent at least bivalent metal ions, including, but not limited to, manganese, copper, iron, barium, strontium, calcium, magnesium, beryllium, chromium, ruthenium, iridium, tantalum, cobalt, nickel, zinc, cadmium, mercury, aluminum, lead, titanium, uranium, gadolinium, platina, gold and silver ions.

The material to which the sequestering agent is added may for example be sediments or sludge, liquid material or liquid material comprising or sediments or sludge.

The methods of the present disclosure further comprise recovering said sequestering agent from said complex. Recovering the sequestering agents enables reuse of the sequestering agent and may thus lead to a decreased amount of sequestering agents being released to the environment.

The methods of the present disclosures are based on the insight that the sequestering agents according to the present disclosure may be recovered, which may decrease the amount of sequestering agent that is released to the environment.

Removal of metal ions from pulp fibers may be performed prior to bleaching of the pulp fibers.

The precipitated metal ions may be disposed of or may be reused in other industrial processes. This means that after complexing metal ions, the complex may be separated from the liquid. This means that that the sequestering agent may be recovered and the metal ions may be disposed of, reused or stored (as complexes) for further processing later. Consequently, the methods of the present disclosure may provide for a decreased amount of sequestering agents and metal ions being released to the environment.

Further, the methods of the present disclosure may be used to enrich metal ions, that occur is diluted liquids, which would be particularly useful for reuse of metal ions of economic interest.

Sequestering agents according to the present disclosure may be suitable for sequestering ions such as manganese, copper, iron, barium, strontium, calcium, magnesium, beryllium, chromium, ruthenium, iridium, tantalum, cobalt, nickel, zinc, cadmium, mercury, aluminum, lead, titanium, uranium, gadolinium, platina, gold and silver ions in applications such as bleaching of cellulose materials such as paper pulps and textiles, varnishing, painting, galvanizing, coating and decontamination of soil, soil leachates and in mining processes.

Moreover, the sequestering agents of the present disclosure may be suitable for sequestering arsenic ions, such as arsenic cations, in aqueous solutions.

The sequestering agents according to the present disclosure may form complex with at least one metal ion, such as two metal ions, i.e. each molecule of sequestering agent may bind at least one metal ion, such as two metal ions. If the sequestering agent may bind two metal ions that are of the same metal or of different metals.

Further, there is provided sequestering agents useful in the methods as described in the first aspect of the disclosure.

Consequently, in another aspect of the disclosure, there is provided a method according to the first aspect, wherein said sequestering agent is represented by formula (I)

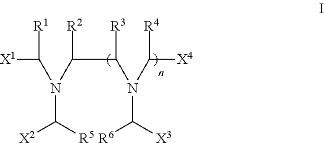

wherein each of R¹, R², R³, R⁴, R⁵ and R⁶ independently is selected from hydrogen and a straight or branched, saturated or unsaturated hydrocarbon chain having from 9 to 20 carbon atoms, and optionally one or two heteroatoms;
n represents 0, 1 or 3;
X¹, X², X³ and X⁴ is independently selected from hydrogen, —CO₂H, —PO₃H₂, —SO₃H, CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷;
R⁷ represents a straight or branched, saturated or unsaturated hydrocarbon chain having from 9 to 20 carbon atoms, wherein 1 or 2 carbon atoms are optionally substituted with one or two heteroatoms;
provided that at least one of R¹, R², R³, R⁴, R⁵ and R⁶ represents said hydrocarbon chain; or if R¹, R², R³, R⁴, R⁵ and R⁶ represents hydrogen, at least one of X¹, X², X³ and X⁴ represents CO₂R⁷, —CONHR⁷, —CH₂OR⁷, —COR⁷, —CH₂OCOR⁷, —CH₂OCONHR⁷, —PO₃HR⁷, —PO₃(R⁷)₂ or —SO₃R⁷; and salts, stereoisomers and mixtures thereof.

In one configuration of this aspect, said sequestering agent is represented by formula (I), wherein n is 0, and X¹ and X² are independently selected from —CO₂H, —PO₃H₂ and —SO₃H.

In another configuration of this aspect, said sequestering agent is represented by formula (I), wherein n is 1, and X¹, X², X³ and X⁴ are independently selected from —CO₂H, —PO₃H₂ and —SO₃H.

In another configuration of this aspect, said sequestering agent is represented by formula (I), wherein at least one of R¹, R², R³, R⁴, R⁵ and R⁶ represents a straight hydrocarbon chain having 12 carbon atoms.

In another configuration of this aspect, said sequestering agent is represented by formula (I), wherein R¹, R², R³, R⁴, R⁵ and R⁶ represents hydrogen; at least one of X¹, X², X³ and X⁴ is independently selected from CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷; and the remaining X¹, X², X³ and X⁴ is independently selected from —CO₂H, —PO₃H₂, and —SO₃H. Preferably, R⁷ represents a straight hydrocarbon chain having 12 carbon atoms.

In another configuration of this aspect, said sequestering agent is selected from

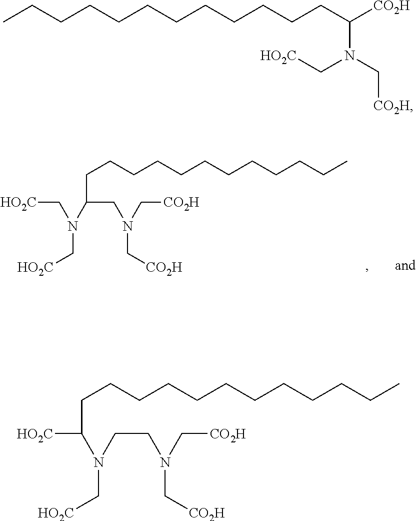

In a another aspect of the disclosure, there is provided a method according to the first aspect, wherein said sequestering agent is represented by formula (II)

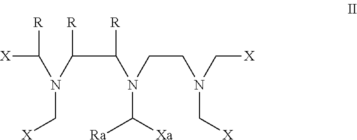

wherein each R and Ra represents hydrogen, or wherein R in one or two positions represents a straight or branched, saturated or unsaturated hydrocarbon chain having from 9 to 20 carbon atoms, and optionally one or two heteroatoms, and the remaining R represents hydrogen;
X and Xa in at least four positions is independently selected from —PO₃H₂, —SO₃H, —PO₃HR⁷, —PO₃(R⁷)₂ and —SO₃R⁷ and the remaining X represents hydrogen;
R⁷ represents a straight or branched, saturated or unsaturated hydrocarbon chain having from 9 to 20 carbon atoms, wherein 1 or 2 carbon atoms are optionally substituted with one or two heteroatoms;
provided that when each R represents hydrogen, at least one X is independently selected from —PO₃HR⁷, —PO₃(R⁷)₂ and —SO₃R⁷ and the remaining X is independently selected from —PO₃H₂ and —SO₃H; and
salts, stereoisomers and mixtures thereof.

In a another aspect of the disclosure, there is provided a method according to the first aspect, wherein said sequestering agent is represented by formula (III)

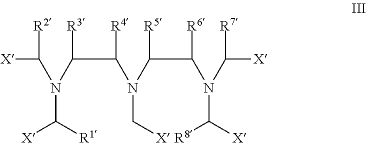

wherein any pair of R^{1′} and R^{2′}; R^{1′} and R^{5′}; R^{1′} and R^{6′}; R^{1′} and R^{7′}; R^{3′} and R^{5′}; R^{3′} and R^{6′}; or R^{4′} and R^{5′} each represents a straight or branched, saturated or unsaturated hydrocarbon chain having from 9 to 20 carbon atoms, and optionally one or two heteroatoms, and the remaining R^{1′}, R^{2′}, R^{3′}, R^{4′}, R^{5′}, R^{6′}, R^{7′} or R^{8′} represents hydrogen;
X′ in each position is independently selected from —CO₂H, —PO₃H₂ and —SO₃H; and
salts, stereoisomers and mixtures thereof.

In one configuration of this aspect, said sequestering agent is represented by formula (III), wherein each of R^{3′} and R^{6′} represents a straight hydrocarbon chain having from 12 carbon atoms and each X′ represents —CO₂H.

In another configuration of this aspect, said sequestering agent is represented by

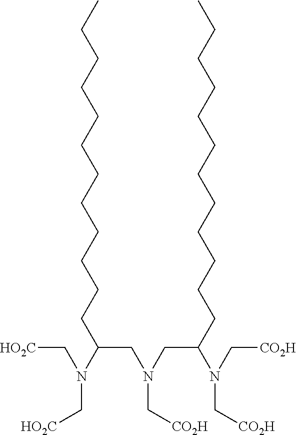

In another aspect of the disclosure, there is provided a method according to the first aspect, wherein said sequestering agent is represented by formula (IV)

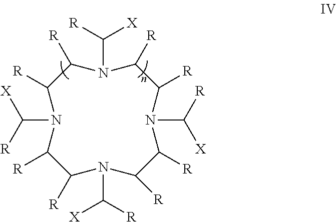

wherein each R represents hydrogen or, in one or two positions R represents a straight or branched, saturated or unsaturated hydrocarbon chain having from 9 to 20 carbon atoms, and optionally one or two heteroatoms, and the remaining R represents hydrogen;
X in at least three or four positions are independently selected from —CO₂H, —PO₃H₂ and —SO₃H and the remaining X represents hydrogen;
n represents 0, 1 or 2;
provided that when each R represents hydrogen, at least one X is independently selected from —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷;
R⁷ represents a straight or branched, saturated or unsaturated hydrocarbon chain having from 9 to 20 carbon atoms, wherein 1 or 2 carbon atoms are optionally substituted with one or two heteroatoms; and salts, pure stereoisomers and mixtures thereof.

In one configuration of this aspect, said sequestering agent is represented by formula (IV), wherein n represents 1; and said R⁷ represents a straight hydrocarbon chain having 12 carbon atoms.

In another configuration of this aspect, said sequestering agent is represented by

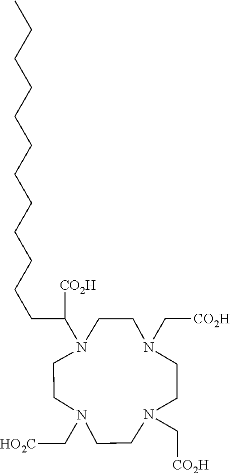

In another aspect of the disclosure, there is provided a method according to the first aspect, wherein said sequestering agent is represented by formula (V)

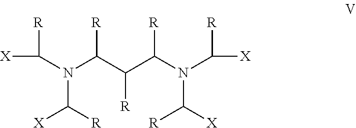

wherein each R represents hydrogen or, in one or two positions R represents a straight or branched, saturated or unsaturated hydrocarbon chain having from 9 to 20 carbon atoms, and optionally one or two heteroatoms, and the remaining R represents hydrogen;
X in at least three positions are independently selected from —CO₂H, —PO₃H₂, —SO₃H, —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X represents hydrogen;
provided that when each R represents hydrogen, at least one X is independently selected from —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X group(s) is independently selected from —CO₂H,

### —PO₃H₂ and —SO₃H;

R⁷ represents a straight or branched, saturated or unsaturated hydrocarbon chain having from 9 to 20 carbon atoms, wherein 1 or 2 carbon atoms are optionally substituted with one or two heteroatoms; and salts, pure stereoisomers and mixtures thereof.

In one configuration of this aspect, said sequestering agent is represented by formula (V), wherein R⁷ represents a straight hydrocarbon chain having 12 carbon atoms.

In another aspect of the disclosure, there is provided a method according to the first aspect, wherein said sequestering agent is represented by formula (VI)

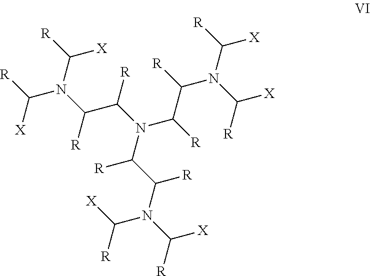

wherein each R represents hydrogen or, in one or two positions R represents a straight or branched, saturated or unsaturated hydrocarbon chain having from 9 to 20 carbon atoms, and optionally one or two heteroatoms, and the remaining R represents hydrogen;
X in at least four positions are independently selected from —CO₂H, —PO₃H₂, —SO₃H, —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X represents hydrogen;
provided that when each R represents hydrogen, at least one X is independently selected from —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X group(s) is independently selected from —CO₂H,

### —PO₃H₂ and —SO₃H;

R⁷ represents a straight or branched, saturated or unsaturated hydrocarbon chain having from 9 to 20 carbon atoms, wherein 1 or 2 carbon atoms are optionally substituted with one or two heteroatoms; and salts, stereoisomers and mixtures thereof.

In one configuration of this aspect, said sequestering agent is represented by formula (VI), wherein R⁷ represents a straight hydrocarbon chain having 12 carbon atoms.

In another aspect of the disclosure, there is provided a method according to the first aspect, wherein said sequestering agent is represented by formula (VII)

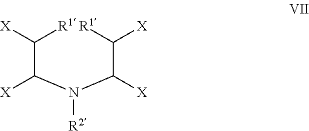

wherein each R^{1′} represents hydrogen or, in one or two positions R^{1′} represents a straight or branched, saturated or unsaturated hydrocarbon chain having from 9 to 20 carbon atoms, and optionally one or two heteroatoms, and the remaining R^{1′} represents hydrogen;
R^{2′} corresponds to R^{1′} or is independently selected from —COR^{1′}, —CH₂CO₂H, —CH₂PO₃H₂ and —CH₂SO₃H;
X in at least three positions is independently selected from —CO₂H, —PO₃H₂ and —SO₃H and the remaining X represents hydrogen;
provided that when R^{1′} represents hydrogen in all positions, X in at least one position is independently selected from —CO₂R^{1′}, —CONHR^{1′}, —CH₂OR^{1′}, —CH₂OCOR^{1′}, —CH₂OCONHR^{1′}, —PO₃HR^{1′}, —PO₃(R^{1′})₂ and —SO₃R^{1′}; R^{2′} is independently selected from —COR^{1′}, —CH₂CO₂R^{1′}, —CH₂CONHR^{1′}, —CH₂CH₂OR^{1′}, —CH₂COR^{1′}, —CH₂CH₂OCOR^{1′}, —CH₂CH₂OCONHR^{1′}, —CH₂PO₃HR^{1′}, —CH₂PO₃(R^{1′})₂, —CH₂SO₃R^{1′}, —CHR^{1′}CO₂H, —CHR^{1′}PO₃H₂, —CHR^{1′}SO₃H, —CH₂CO₂H, —CH₂PO₃H₂ and —CH₂SO₃H; and the remaining positions of X are independently selected from —CO₂H, —PO₃H₂ and —SO₃H; and salts, stereoisomers and mixtures thereof.

In another aspect of the disclosure, there is provided a method according to the first aspect, wherein said sequestering agent is represented by formula (VIII)

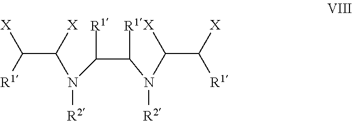

wherein each R^{1′} represents hydrogen or, in one or two positions R^{1′} represents a straight or branched, saturated or unsaturated hydrocarbon chain having from 9 to 20 carbon atoms, and optionally one or two heteroatoms, and the remaining R^{1′} represents hydrogen;
each R^{2′} represents hydrogen or, in one or two positions R^{2′} represents a straight or branched, saturated or unsaturated hydrocarbon chain having from 9 to 20 carbon atoms, and optionally one or two heteroatoms, or in at least one position R^{2′} is independently selected from —COR^{2′}, —CH₂CO₂H, —CH₂PO₃H₂ and —CH₂SO₃H;
X in at least three positions is independently selected from —CO₂H, —PO₃H₂, —SO₃H, —CO₂R^{1′}, —CONHR^{1′}, —CH₂OR^{1′}, —CH₂OCOR^{1′}, —CH₂OCONHR^{1′}, —PO₃HR^{1′}, —PO₃(R^{1′})₂ and —SO₃R^{1′} and the remaining X represents hydrogen;
provided that when R^{1′} represents hydrogen in all positions, X in at least one position is independently selected from —CO₂R^{1′}, —CONHR^{1′}, —CH₂OR^{1′}, —COR^{1′}, —CH₂OCOR^{1′}, —CH₂OCONHR^{1′}, —PO₃HR^{1′}, —PO₃(R^{1′})₂ and —SO₃R^{1′}; or R^{2′} is independently selected from —COR^{2′}, —CH₂CO₂R2^{1′}, —CH₂CONHR^{2′}, —CH₂CH₂OR^{2′}, —CH₂COR^{2′}, —CH₂CH₂OCOR^{2′}, —CH₂CH₂OCONHR^{2′}, —CH₂PO₃HR², —CH₂PO₃(R^{2′})₂, —CH₂SO₃R^{2′}, —CHR^{2′}CO₂H, —CHR^{2′}PO₃H₂, —CHR^{2′}SO₃H, —CH₂CO₂H, —CH₂PO₃H₂ and —CH₂SO₃H, wherein R^{2′} represents a straight or branched, saturated or unsaturated hydrocarbon chain having from 9 to 20 carbon atoms; and
salts, pure stereoisomers and mixtures thereof.

In one configuration of this aspect, said sequestering agent is represented by

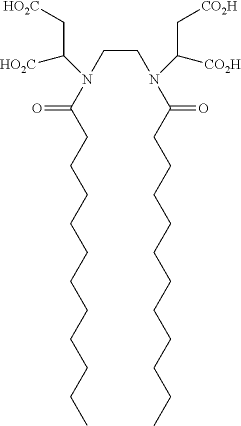

In another aspect of the disclosure, there is provided a method according to the first aspect, wherein said sequestering agent is selected from 2-dodecyl-3-carboxymethyl-3-azapentane diacid, 2-dodecyl-3,6-di(carboxymethyl)-3,6-diazaoctane diacid and 4-dodecyl-3,6-di(carboxymethyl)-3,6-diazaoctane diacid. These compounds have excellent sequestering properties, as illustrated by the Examples of the present disclosure.

In yet another aspect of the disclosure, there is provided a sequestering agent represented by formula (I)

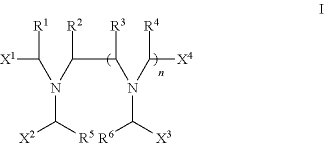

wherein each of R¹, R², R³, R⁴, R⁵ and R⁶ independently is selected from hydrogen and a straight or branched, saturated or unsaturated hydrocarbon chain having from 9 to 20 carbon atoms, and optionally one or two heteroatoms, provided that at least one of R¹, R², R³, R⁴, R⁵ and R⁶ represents said hydrocarbon chain;
n represents 0, 1 or 3;
X¹, X², X³ and X⁴ is independently selected from hydrogen, —CO₂H, —PO₃H₂, —SO₃H, CO₂R⁷, —CONHR⁷, —CH₂OR⁷, —COR⁷, —CH₂OCOR⁷,

### —CH₂OCONHR⁷, —PO₃HR⁷, —PO₃(R⁷)₂ and —SO₃R⁷;

R⁷ represents a straight or branched, saturated or unsaturated hydrocarbon chain having from 9 to 20 carbon atoms, wherein 1 or 2 carbon atoms are optionally substituted with one or two heteroatoms;
provided that when n is 0; X¹, X² and X⁴ is selected from —PO₃H₂ and —SO₃H; and
provided that when n is 1, at least three of X¹, X², X³ and X⁴ is selected from —CO₂H, —PO₃H₂ and —SO₃H; and
provided that when n is 1; X¹, X², X³ and X⁴ represents —CO₂H; R², R³, R⁴, R⁵ and R⁶ represents hydrogen; then R¹ is not a straight hydrocarbon chain having 10 or 14 carbon atoms; and provided that when n is 1; X¹, X², X³ and X⁴ represents —CO₂H; R¹, R³, R⁴, R⁵ and R⁶ represents hydrogen; then R² is not a straight hydrocarbon chain having 10, 12 or 14 carbon atoms; and
provided that when n is 1; X¹, X², X³ and X⁴ represents —CO₂H; R², R³, R⁵ and R⁶ represents hydrogen; then R¹ and R⁴ is not a straight hydrocarbon chain having 10 or 12 carbon atoms; and R¹ is not a straight hydrocarbon chain having 10 carbon atoms and R⁴ is not a straight hydrocarbon chain having 12 carbon atoms at the same time; and
provided that when n is 1; X¹, X², X³ and X⁴ represents —CO₂H; R¹, R⁴, R⁵ and R⁶ represents hydrogen; then R² and R³ is not a straight hydrocarbon chain having 10 or 12 carbon atoms; and R² is not a straight hydrocarbon chain having 10 carbon atoms and R³ is not a straight hydrocarbon chain having 12 carbon atoms at the same time; and
provided that when n is 1; X², X³ and X⁴ represents —CO₂H; and X¹ represents CH₂CONR⁷, then R⁷ is not a straight hydrocarbon chain having 10, 12 or 14 carbon atoms; and
provided that when n is 1; X², X³ and X⁴ represents —CO₂H; and X¹ represents CH₂CO₂R⁷, then R⁷ is not a straight hydrocarbon chain having 10, 12, 14, 16 or 18 carbon atoms; or when X¹ represents CH₂OCOR⁷, then R⁷ is not a straight hydrocarbon chain having 17 carbon atoms; and salts, stereoisomers and mixtures thereof.

In one configuration of this aspect, there is provided a sequestering agent represented by formula (I) wherein n is 0; and R², R⁵ and R⁶ represents hydrogen.

In another configuration of this aspect, there is provided a sequestering agent represented by formula (I) wherein X¹, X² and X³ represents —CO₂H.

In another configuration of this aspect, there is provided a sequestering represented by formula (I) wherein R¹ represents a straight hydrocarbon chain having 12 carbon atoms.

In another configuration of this aspect, there is provided a sequestering agent represented by formula (I) wherein n is 1; and R³, R⁴, R⁵ and R⁶ represents hydrogen.

In another configuration of this aspect, there is provided a sequestering agent represented by formula (I) wherein X¹, X², X³ and X⁴ represents —CO₂H.

In another configuration of this aspect, there is provided a sequestering agent represented by formula (I) wherein R¹ represents a straight hydrocarbon chain having 12 carbon atoms and R² represents hydrogen; or wherein R² represents a straight hydrocarbon chain having 12 carbon atoms and R¹ represents hydrogen.

In another configuration of this aspect, there is provided a sequestering agent represented by formula (I) wherein said agent is selected from

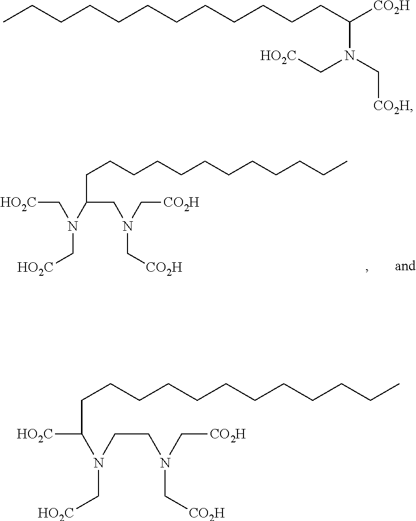

In yet another aspect of the disclosure, there is provided a sequestering agent represented by formula (II)

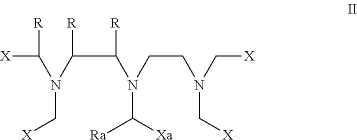

wherein each R and Ra represents hydrogen, or wherein R in one or two positions represents a straight or branched, saturated or unsaturated hydrocarbon chain having from 9 to 20 carbon atoms, and optionally one or two heteroatoms, and the remaining R represents hydrogen;
X and Xa in at least four positions is independently selected from —PO₃H₂, —SO₃H, —PO₃HR⁷, —PO₃(R⁷)₂ and —SO₃R⁷ and the remaining X represents hydrogen;
R⁷ represents a straight or branched, saturated or unsaturated hydrocarbon chain having from 9 to 20 carbon atoms, wherein 1 or 2 carbon atoms are optionally substituted with one or two heteroatoms;
provided that when each R represents hydrogen, at least one X is independently selected from —PO₃HR⁷, —PO₃(R⁷)₂ and —SO₃R⁷ and the remaining X is independently selected from —PO₃H₂ and —SO₃H; and provided that when each X represents —PO₃H₂; Xa and R represents H; then Ra is not a hydrocarbon chain having 12 or 16 carbon atoms; and salts, stereoisomers and mixtures thereof.

In yet another aspect of the disclosure, there is provided a sequestering agent represented by formula (III)

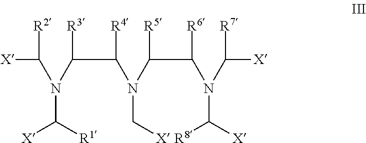

wherein any pair of R^{1′} and R^{2′}; R^{1′} and R^{5′}; R^{1′} and R^{6′}; R^{1′} and R^{7′}; R^{3′} and R^{5′}; R^{3′} and R^{6′}; or R^{4′} and R^{5′} each represents a straight or branched, saturated or unsaturated hydrocarbon chain having from 9 to 20 carbon atoms, and optionally one or two heteroatoms, and the remaining R^{1′}, R^{2′}, R^{3′}. R^{4′}, R^{5′}, R^{6′}, R^{7′} or R^{8′} represents hydrogen;
X′ in each position is independently selected from —CO₂H, —PO₃H₂ and —SO₃H; and
salts, stereoisomers and mixtures thereof.

In one configuration of this aspect, there is provided a sequestering represented by formula (II), wherein each of R^{3′} and R^{6′} represents a straight hydrocarbon chain having from 12 carbon atoms and each X′ represents —CO₂H.

In another configuration of this aspect, there is provided a sequestering agent represented by formula (II), represented by

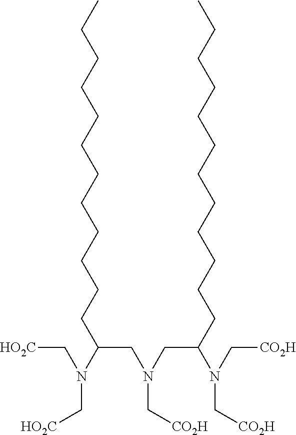

In yet another aspect of the disclosure, there is provided a sequestering agent represented by formula (IV)

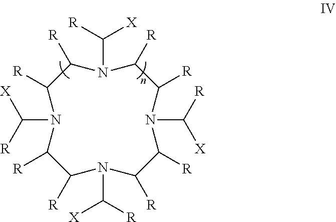

wherein each R represents hydrogen or, in one or two positions R represents a straight or branched, saturated or unsaturated hydrocarbon chain having from 9 to 20 carbon atoms, and optionally one or two heteroatoms, and the remaining R represents hydrogen;
X in at least three or four positions are independently selected from —CO₂H, —PO₃H₂, —SO₃H—CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷;
R⁷ represents a straight or branched, saturated or unsaturated hydrocarbon chain having from 9 to 20 carbon atoms, wherein 1 or 2 carbon atoms are optionally substituted with one or two heteroatoms;
n represents 0, 1 or 2; and salts, stereoisomers and mixtures thereof;
provided that when n represents 1, the following compounds are excluded:

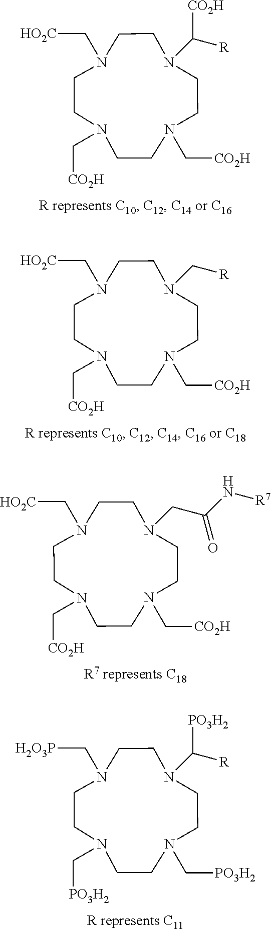

In yet another aspect of the disclosure, there is provided a sequestering agent represented by formula (Va)

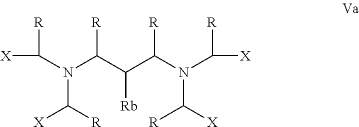

wherein each R and Rb represents hydrogen or, in one or two positions of R or Rb, represents a straight or branched, saturated or unsaturated hydrocarbon chain having from 9 to 20 carbon atoms, and optionally one or two heteroatoms, and the remaining R represents hydrogen;

X is independently selected from —CO₂H, —PO₃H₂ and —SO₃H;
provided that when each R represents hydrogen, at least one X is independently selected from —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X group(s) is independently selected from —CO₂H,

### —PO₃H₂ and —SO₃H;

R⁷ represents a straight or branched, saturated or unsaturated hydrocarbon chain having from 9 to 20 carbon atoms, wherein 1 or 2 carbon atoms are optionally substituted with one or two heteroatoms;
provided that when each X represents —CO₂H, Rb represents a saturated or unsaturated hydrocarbon chain having from 13 to 20 carbon atoms, and optionally one or two heteroatoms; and salts, stereoisomers and mixtures thereof.

In one configuration of this aspect, there is provided a sequestering agent represented by formula (Va), wherein said R⁷ represents a straight hydrocarbon chain having 12 carbon atoms.

In yet another aspect of the disclosure, there is provided a sequestering agent represented by formula (VI)

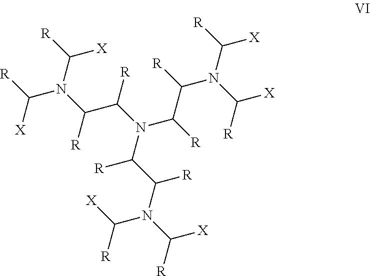

wherein each R represents hydrogen or, in one or two positions R represents a straight or branched, saturated or unsaturated hydrocarbon chain having from 9 to 20 carbon atoms, and optionally one or two heteroatoms, and the remaining R represents hydrogen;
X in at least four positions is independently selected from —CO₂H, —PO₃H₂, —SO₃H, —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X represents hydrogen;
provided that when each R represents hydrogen, at least one X is independently selected from —CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷ and the remaining X group(s) is independently selected from —CO₂H, —PO₃H₂ and —SO₃H;
R⁷ represents a straight or branched, saturated or unsaturated hydrocarbon chain having from 9 to 20 carbon atoms, wherein 1 or 2 carbon atoms are optionally substituted with one or two heteroatoms; and salts, stereoisomers and mixtures thereof.

In one configuration of this aspect, there is provided a sequestering agent represented by formula (VI), wherein said R⁷ represents a straight hydrocarbon chain having 12 carbon atoms.

In yet another aspect of the disclosure, there is provided a sequestering agent represented by formula (VII)

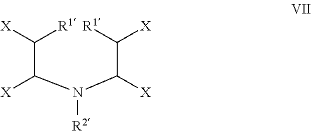

wherein R^{1′} represents hydrogen or, in one or two positions represents a straight or branched, saturated or unsaturated hydrocarbon chain having from 9 to 20 carbon atoms, and optionally one or two heteroatoms, and the remaining R^{1′} represents hydrogen;
R^{2′} corresponds to R^{1′} or is independently selected from —COR^{1′}, —CH₂CO₂H, —CH₂PO₃H₂ and —CH₂SO₃H;
X in at least three positions are independently selected from —CO₂H, —PO₃H₂ and —SO₃H and the remaining X represents hydrogen;
provided that when X represents —CO₂H, then R^{2′} is not a straight hydrocarbon chain having 10, 12, 14, 16 or 18 carbon atoms; and
provided that when X represents —CO₂H and R^{2′} represents —COR^{1′}, then R^{1′} is not a straight hydrocarbon chain having 17 carbon atoms; and salts, stereoisomers and mixtures thereof.

In yet another aspect of the disclosure, there is provided a sequestering agent represented by formula (VIII)

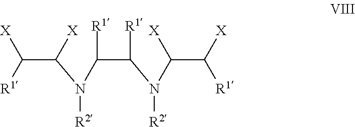

wherein R^{1′} represents hydrogen or, in one or two positions R^{1′} represents a straight or branched, saturated or unsaturated hydrocarbon chain having from 9 to 20 carbon atoms, and optionally one or two heteroatoms, and the remaining R^{1′} represents hydrogen;
R^{2′} corresponds to R^{1′}, or in at least one position independently selected from —COR^{1′}, —CH₂CO₂H, —CH₂PO₃H₂ and —CH₂SO₃H;
X in at least three positions are independently selected from —CO₂H, —PO₃H₂ and —SO₃H and the remaining X represents hydrogen;
provided that when X represents —CO₂H, and R^{2′} represents —COR^{1′}, then R^{1′} is not a straight hydrocarbon chain having 9, 11, 12, 13, 15 or 17 carbon atoms; and salts, stereoisomers and mixtures thereof.

In yet another aspect of the disclosure, there is provided a method according to the first aspect, wherein said sequestering agent is represented by formula (IX)

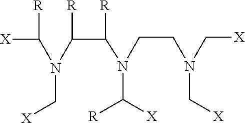

wherein R in at least one of the positions shown is comprised of a group in the form of a straight or branched hydrocarbon chain having from 9 to 20 carbon atoms and eventually 1-2 heteroatoms and which is missing in other position(s);
X in at least four of the positions shown is a group in the form of —COOH or the salt thereof and which in the case of four groups is missing in one position;
wherein the chemical can be a racemate or a mixture in different proportions or pure enantiomers wherein R or X is missing it shall be an H,
or; where R is missing in all four positions shown X in at least one position is —COOR or —CONHR or —CH₂OR or —COR or —CH₂OCOR or CH₂OCONHR; and where X in the remaining of the positions shown is comprised of a group in the form of —COOH or its salt and where the chemical can be a racemate or a mixture in different proportions or pure enantiomers
where R or X is missing it shall be an H.

In one configuration of this aspect, there is provided a sequestering agent represented by formula (IX), wherein R occurs in at least one of the three positions to the left in the structural formula.

In another configuration of this aspect, there is provided a sequestering agent represented by formula (IX), wherein, wherein R occurs in position 2, counted from the left in the structural formula.

In another configuration of this aspect, there is provided a sequestering agent represented by formula (IX), wherein the number of carbon atoms in the hydrocarbon chain of R is 10 to 14.

In another configuration of this aspect, there is provided a sequestering agent represented by formula (IX), the number of carbon atoms in the hydrocarbon chain of R is more than 14 and at most 20.

In another configuration of this aspect, there is provided a sequestering agent represented by formula (IX), wherein R is missing as solitaire in the structural formula the modified X is comprised of —CONHR or —CH₂OR and —COR and preferably —CONHR.

In another configuration of this aspect, there is provided a sequestering agent represented by formula (IX), wherein heteroatoms are meant one or several of the atoms sulphur, oxygen and nitrogen.

In another configuration of this aspect, there is provided a sequestering agent represented by formula (IX), wherein one or more solitaire R occur is (are) placed between the carbon atom in question and the hydrocarbon chain.

In another configuration of this aspect, there is provided a sequestering agent represented by formula (IX), wherein said agent is represented by 4-dodecyl-3,6,9-tri(carboxymethyl)-3,6,9-triazaundecane diacid or its salt.

The sequestering agents of the present disclosure may be selected depending on the application. As an example, sequestering agents having a sidechain comprising at least 14 carbon atoms, such as about 15-20 carbon atoms, may be used if the metal ions are present in a liquid, such as in a lechate. As a further example, sequestering agents having a sidechain comprising about 9-14 carbon atoms, such as 12 carbon atoms, may be used if the metal ion is in a liquid having a high solids content, such as a pulp. For other applications, a combination of one or more sidechain(s) comprising at least 14 carbon atoms and or of one or more sidechain(s) comprising about 9-14 carbon atoms may be useful.

In another aspect of the invention, there is provided a composition comprising at least one sequestering agent according to the present disclosure. As examples, the composition may comprise at least one, such as at least two, such as at least three, such as at least four, sequestering agents according to any configuration of the first aspect. The sequestering agents of the composition may be selected depending on the application, e.g. depending on the type of metal ions present in e.g. the liquid to which the composition is added. Consequently, the composition may comprise a cocktail of sequestering agents in order to sequester different types of metal ions.

In another aspect of the invention, there is provided the use of at least one sequestering agent according to the present disclosure or a composition according to the present disclosure for sequestering at least one metal ion. As an example, at least one sequestering agent according to the present disclosure or a composition according to the present disclosure may be used for sequestering a at least one metal ion selected from manganese, copper, iron, barium, strontium, calcium, magnesium, beryllium, chromium, ruthenium, iridium, tantalum, cobalt, nickel, zinc, cadmium, mercury, aluminum, lead, titanium, uranium, gadolinium, platina, gold and silver ions. It is to be understood that a composition comprising at least one sequestering agent according to the present disclosure may be used in the method according to the present disclosure.

### BRIEF DESCRIPTION OF THE DRAWINGS

FIG. 1 illustrates a set up for flotation of metal ions using sequestering agents of the present disclosure. A description is provided in Exemplary embodiment 1.

FIG. 2 illustrates a set up for flotation of metal ions in a pulping process. A description is provided in Exemplary embodiment 2.

FIG. 3 illustrates a setup for recovery of sequestering agents and metals from agents of the present disclosure. A description is provided in Exemplary embodiment 3.

### EXEMPLARY EMBODIMENTS

The following non-limiting exemplary embodiments will further illustrate the present invention.

### Exemplary Embodiment 1

### Sequestering Metal Ions in a Leachate

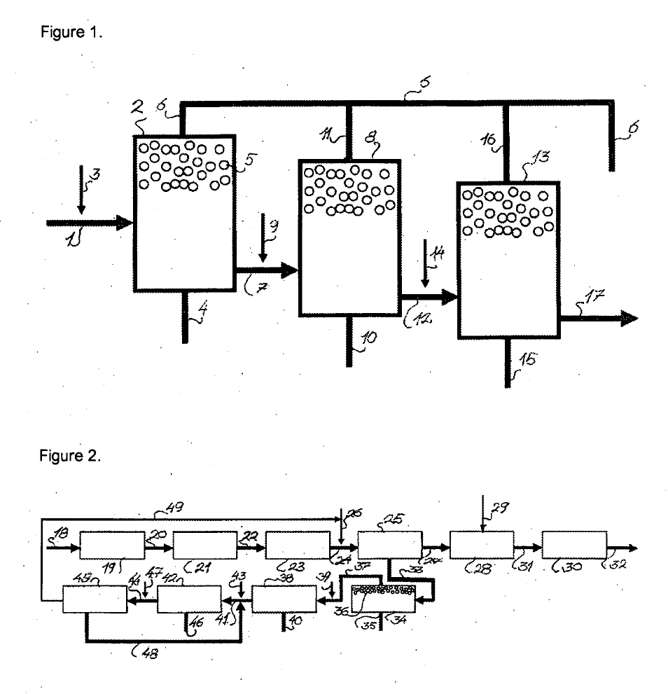

Exemplary embodiment 1 is a non-limiting example in removing metal ions from a leachate using flotation and sequestering agents of the present disclosure. FIG. 1 shows how leachate is transported to the flotation vessel 2 through conduit 1. Through the conduit 3 a sequestering agent according to the present disclosure is added to the leachate together with at least one surfactant, for example a surfactant of the type alkylsulphates, alkylsulphonates, alkylcarboxylates, alkylethoxylates. At the bottom of the flotation vessel 2, air is added through conduit 4, which in the form of gas bubbles 5 that flows upwards in the vessel 2. Alternatively, a stream may be obtained by the use of a rotation means, such as a propeller.

Complex of sequestering agents and metal ions are transported by the gas bubbles 5 to at the top of the vessel 2, forming a foam on, top of the flotation vessel 2. The foam is scraped off from the top surface and is removed through the main conduit 6. The leachate partly relieved from metals is transported through the conduit 7 to a second flotation vessel 8. Through conduit 9 is added further sequestering agents and surfactants. Air is supplied through the conduit 10 and the foam formed is transported through the conduit 11 to the main conduit 6. In a third step leachate is led through the conduit 12 to the flotation vessel 13. Sequestering agents and surfactants are added through the conduit 14 and air through the conduit 15. The foam is removed through the conduit 16 to be introduced into the main conduit 6. Leachate that has been subjected to three flotations is removed from the conduit 17 and formed foam is transported in main conduit 6. However, it is to be understood that the leachate may be subjected to more than three flotations in order to further decrease the concentration of metal ions. Further, one flotation may well be sufficient to obtain a satisfying result.

### Exemplary Embodiment 2

### Sequestering Metal Ions from Cellulose Pulp

Exemplary embodiment 2 is a non-limiting example in sequestering metal ions from cellulose pulp using sequestering agents of the present disclosure. FIG. 2 shows bleaching of a mechanical cellulose pulp with hydrogen peroxide, wherein sequestering agents according to the invention are added to the cellulose pulp for capturing of undesired metals (including manganese ions) in the cellulose pulp before the bleaching step and for recovery of sequestering agents, which are rejected from the cellulose pulp manufacturing process in the form of chelates (complexes). Wood chips are input through the conduit 18 to the refiner 19 wherein the wood chips are converted to cellulose pulp. This is transported through the conduit 20 to a screening department 21. Subsequently the screened and/or hydrocyclone purified cellulose pulp is fed through the conduit 22 to a washing step 23. From this step the cellulose pulp is led through the conduit 24 to a press (or wash press) 25. On the way to the press 25 a sequestering agent according to the invention is added to the cellulose pulp through the conduit 26.

Cellulose pulp with a high pulp concentration is led through the conduit 27 (for example with the aid of a screw conveyor) to a chemical mixer 28, to which bleaching chemicals are added through the conduit 29 in the form of hydrogen peroxide and sodium hydroxide and possibly some further chemicals, such as water glass (Na₂SiO₃). Thereafter, the cellulose pulp is fed into the bleaching tower 30 through the conduit 31. After a bleaching time of a few hours, the bleached cellulose pulp is further led through the conduit 32 to a washing step (not shown in the figure). The liquid resulting in the press 25 (i.e. liquid pressed out from the cellulose pulp suspension), which has a content of chelate (complex) of sequestering agent:metal ion, is led through the conduit 33 to a flotation vessel 34. Through the conduit 35 air is added to the flotation vessel 34, and air flows upwards in the vessel in the form of bubbles 36. As described in Example 1 above, a foam comprising the complex is formed at the top of the flotation vessel. The foam is removed/separated from the top surface of the liquid column and is transported through the conduit 37 to the acid treatment vessel 38. The purified, i.e. pressed material that has been subjected to flotation, is fed out of the flotation vessel 34 for a possible completing treatment (not shown in the figure).

Since the cellulose pulp fibers give away fatty acids and resinous acids to the pressate, it may not be necessary to add any aiding flocculating agent, such as a surfactant, to the flotation vessel 34. However a flocculating agent may be added to aid the flotation process. Addition of surfactants may depend on the sequestering agent used.

Through the conduit 39 an acid is added to the possibly collapsed foam, such as a mineral acid or carbonic acid. Enough acid is added to decrease the pH-value of the formed liquid to about 0-3, which precipitates metal ions complexed with the sequestering agents. Further, the complexes are separated in the vessel 38 from fatty acids, resinous acids and the metal ions. Surfactants may be removed from the vessel 38 through the conduit 40, while the complexes are led through the conduit 41 to the extraction vessel 42. Heptane is added as an extraction agent through the conduit 43. The sequestering agent molecules are converted from the water phase to the solvent phase and this is led through conduit 44 to the dwell vessel 45. The water phase with its content of diverse chemicals is ejected from the system through the conduit 46.

An alkaline aqueous solution of such a strength and in such an amount that the pH-value in the water phase becomes at least 7 is added to the solvent phase containing the sequestering agent in conduit 47. Hereby, the sequestering agent will move from the solvent phase over to the aqueous phase. These two phases are separated from each other and the solvent phase is returned into the system through the conduit 48 at input-position 43. The aqueous phase containing the recovered sequestering agent is returned into the system through the conduit 49 at input-position 26.

Since the solvent as well as the sequestering agent is recovered the conduits 26 and 43 symbolize only addition of fresh, non-used sequestering agent and heptanes, respectively. The fresh addition of these chemicals may be limited and correspond to the spillage occurring in the system for the respective chemical. It is further to be understood that the method above may be modified such that separation of the chelate formed between sequestering agent and metal ions may be performed after bleaching of the pulp. This could be carried out in two or more steps.

### Exemplary Embodiment 3

### Recovering Sequestering Agents and Metals from Complexes

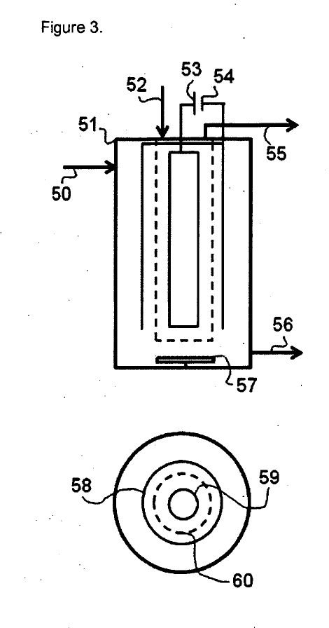

Exemplary embodiment 3 is a non-limiting example describing recovery of sequestering agents and metals from agents of the present disclosure. FIG. 3 illustrates how an aqueous electrolyte solution consisting of sodium sulfate (Na₂SO₄) in a concentration range of typically 0.001 to 1 M is transported to the anodic compartment of the electrolysis vessel 51 through conduit 50. Through the conduit 52, a foam fraction consisting of the complexes of sequestering agents and metal ions from conduit 6 in FIG. 1 or conduits 37, 41 or 49 in FIG. 2, is fed to the cathodic compartment of the electrolysis vessel 51. A direct current (DC) voltage supply with its negative output 53 connected to the cathode 59, and its positive output 54 connected to the anode 58, is used and a voltage of typically between 1.5 and 20 V is applied. A semi-permeable membrane 60, especially constructed for retaining larger molecules than simple salt ions, is used as a separator between the solutions at the anode and the cathode. A propeller 57 is used for decreasing the electrolyte concentration gradients in the electrolysis vessel and to increase the transport of ions through the semi-permeable membrane 60. After sufficient electrolysis time, typically between 10 to 60 min depending on the applied current and the concentration of complexes of sequestering agents and metal ions, the metal is collected as a solid covering the cathode 59, and the solution containing the sequestering agent is transported through conduit 55 for later re-use. If necessary, the electrolyte solution can be fed out through conduit 56.

### EXAMPLES

The following non-limiting experimental examples will further illustrate the present invention.

### Experimental Example 1

### Sequestering of metal ions using 2-dodecyl-3,6-di(carboxymethyl)-3,6-diazaoctane diacid

### Materials and Methods

In order to investigate the separability of a sequestering agent according to the invention a small flotation cell was used. This flotation cell has a volume of approximately 1.6 l, a height of 315 mm and an inner diameter of 80 mm. Compressed air used to form the foam is led through a porous sintered glass filter of diameter 60 mm with a nominal porosity of 10-16 μm (“porosity 4”) mounted at the bottom of the flotation cell. At the top of the flotation cell a cylinder of an inner diameter of 30 mm and a height of 415 mm, with an outlet placed at 72 mm from the bottom, is mounted. The outlet is used to collect the foam and thereby the chelate according to the invention. At the top of the latter cylinder an adjustable valve is mounted to be able to better control the foaming and to direct the foam to the outlet.

A sequestering agent, 2-dodecyl-3,6-di(carboxymethyl)-3,6-diazaoctane diacid,

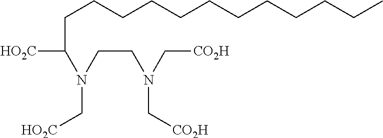

was prepared from 2-aminoethanol, tert-butyl bromoacetate and tert-butyl 2-aminotetradecanoate as main ingredients using conventional techniques and was therafter mixed in 500 ml deionised water with 1 mg of manganese in the form of manganese sulphate (in a molar ratio of 1.2:1=sequestering agent: manganese sulphate) and a flotation agent (N,N-dimethyldodecylamine N-oxide, in a molar ratio of 10:1=flotation agent:sequestering agent).

The pH-value of the solution was adjusted to pH 5.5 with 0.1 M sodium hydroxide solution or 0.1 M hydrogen chloride solution. The solution was carefully stirred in 30 min for equilibration. Thereafter the solution was transferred to the earlier described flotation cell. Deionised water (pH adjusted to 5.5) was added to a total volume of 1000 ml. Air flow to the flotation cell was turned on leading to the formation of gas (air) bubbles which rose upwards in the cell. Foam was collected (36.3 g) until the foam formation decreased to a minimum (approximately 30 min.). The foam was taken for manganese analysis. The same experiment as above was also performed with 1 mg of copper in form of copper sulphate.

### Results

Metal analyses were made with the aid of a Perkin-Elmer 3110 atomic absorption spectrometer according a standardize method for metal analyzes; SCAN-CM 38:05. About 35% of the added manganese or 65% of the added copper were found in the foam, where the manganese or copper were bonded to the added sequestering agent according to the invention. The concentration of manganese or copper in the foam was about ten times higher than the concentration of manganese or copper in the solution before the flotation.

Thus, this example showed that 2-dodecyl-3,6-di(carboxymethyl)-3,6-diazaoctane diacid worked excellent as a sequestering agent.

### Experimental Example 2

### Sequestering of metal ions using 2-dodecyl-3-carboxymethyl-3-azapentane diacid

### Materials and Methods

A sequestering agent, 2-dodecyl-3-carboxymethyl-3-azapentane diacid,

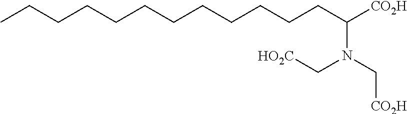

was prepared from 2-aminotetradecanoic acid an ethyl bromoacetic acid as main ingredients using conventional techniques and was therafter mixed in 500 ml deionised water with 1 mg of manganese in the form of manganese sulphate (in a molar ratio of 1.2:1=sequestering agent: manganese sulphate) and a flotation agent (N,N-dimethyldodecylamine N-oxide, in a molar ratio of 10:1=flotation agent:sequestering agent). The pH-value of the solution was adjusted to pH 5.5 with 0.1 M sodium hydroxide solution or 0.1 M hydrogen chloride solution. The solution was carefully stirred in 30 min. for equilibration. Thereafter the solution was transferred to flotation cell described in Experimental Example 1. Deionised water (pH adjusted to 5.5) was added to a total volume of 1000 ml. Air flow to the flotation cell was turned on leading to the formation of gas (air) bubbles which rose upwards in the cell. Foam was collected (54.5 g) until the foam formation decreased to a minimum (approximately 30 min.). The foam was taken for manganese analysis. The same experiment as above was also performed with 1 mg of copper in form of copper sulphate.

### Results

Metal analyses were made with the aid of a Perkin-Elmer 3110 atomic absorption spectrometer according a standardize method for metal analyzes; SCAN-CM 38:05. About 10% of the added manganese or 70% of the added copper were found in the foam, where the manganese or copper were bonded to the added sequestering agent according to the invention. The concentration of manganese in the foam was about two times higher or the concentration of copper in the foam was about thirteen times higher than the concentration of manganese or copper, respectively, in the solution before the flotation.

Thus, this example showed that 2-dodecyl-3-carboxymethyl-3-azapentane diacid worked excellent as a sequestering agent.

### Experimental Example 3

### Sequestering of Metal Ions in Thermomechanical Pulp

### Materials and Methods

The cellulose pulp was removed directly after the refiner in a TMP-plant and its dry solids content was determined with the aid of “Mettler Toledo HR 73 Halogen Moisture Analyzer”. 70 g bone-dry cellulose pulp was then slushed in 1.4 l cold distilled water with the aid of a slusher of model “Lorentzon & Wettre App. 03, type 8-3, no. 723”. The cellulose pulp with a concentration of 4.8 percent by weight was filtered on a Büchner funnel and the filtrate was returned to be filtered again.

Thereafter the cellulose pulp was slushed in 1.4 l distilled water at a temperature of 55° C. The pulp suspension was left to stand for 1 h and was then filtered two times according to the same process being described above. Again the cellulose pulp was slushed in 1.4 l distilled water at a temperature of 55° C.

A sequestering agent according to the present invention, 2-dodecyl-3,6-di(carboxymethyl)-3,6-diazaoctane diacid

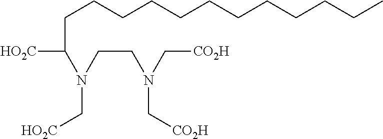

was prepared as described in Example 1 and added to a portion of the pulp. As a comparison, the conventional sequestering agent DTPA (diethylenetriaminepentaacetic acid) was added to another portion of the pulp. The added amount of sequestering agent was 0.17 mmol, corresponding to a molar ratio of manganese/sequestering agent of 1:1.3 at an anticipated manganese content in the cellulose pulp of 100 ppm. The pH was measured in the pulp suspension and it amounted to 6.2 and the cellulose pulp suspension was allowed to stand, i.e. the sequestering agent was allowed to work for a time of 60 min. Thereafter the formed chelate was removed from the cellulose pulp by filtration of the same in the above described way. The manganese content of the cellulose pulp was determined, on one hand, on non-treated pulp, and on the other hand on the portions having been treated with the respective sequestering agents according to the following: 1 g of bone-dry cellulose pulp was transferred to a Teflon-lined vessel specially designed for microwave oven digestion (Microwave Accelerated Reaction System, MARS 5, CEM). 12 ml of 65% HNO₃ (p.A.) was added and the pulp sample was stirred. The sample was treated in the microwave, which was programmed to increase the effect ramp-wise to 600 W during 25 min, without exceeding a pressure of 650 psi, where after constant pressure and effect was maintained during 5 min. After cooling, the sample solution was analyzed in view of among other things manganese content according to a standardized method for metal analyzes; SCAN-CM 38:05, using a Perkin-Elmer 3110 atomic absorption spectrometer.

### Results

The starting cellulose pulp had a manganese content of 104 mg/kg. The portion of the pulp treated with 2-dodecyl-3,6-di(carboxymethyl)-3,6-diazaoctane diacid and further releaved from the chelates had a manganese content of 6.2 mg/kg, whereas the portion of the pulp treated DTPA and further releaved from chelates had a manganese content of 9.3 mg/kg.

Thus, this example showed that a sequestering agent according to the present invention could remove a larger amount of manganese ions from thermomechanical pulp (TMP) manufactured from spruce compared to the conventional sequestering agent DTPA.

### Experimental Example 4

### Solubility of 4-dodecyl-3,6,9-tri(carboxymethyl)-3,6,9-triazaundecane diacid in a copper(II) chloride solution

### Materials and Methods

A stock solution of 4-dodecyl-3,6,9-tri(carboxymethyl)-3,6,9-triazaundecane diacid and copper in the form of copper(II) chloride (20 ml, in a molar ratio of 1.2:1=sequestering agent: copper(II) chloride, [Cu²⁺]=900 ppm, pH=4.5) was diluted with milliQ-water to a total volume of 60 ml in a beaker, corresponding to a solution with an initial sequestrent and copper concentration of 5.66 mM and 300 ppm, respectively. To this solution 2 M aqueous hydrogen chloride was added stepwise, under magnetical stirring at room temperature, to follow the solubility behavior of 4-dodecyl-3,6,9-tri(carboxymethyl)-3,6,9-triazaundecane diacid.

### Results

Observations at different pH intervals.

pH=4.5-2.8: A clear blue aqueous solution.
pH=2.8-0.5: Precipitated material is observed.
pH<0.5: A clear blue aqueous solution.

### Experimental Example 5

### Precipitation/recovery of 4-dodecyl-3,6,9-tri(carboxymethyl)-3,6,9-triazaundecane diacid from a copper(II) chloride solution

### Materials and Methods

A stock solution of 4-dodecyl-3,6,9-tri(carboxymethyl)-3,6,9-triazaundecane diacid and copper in the form of copper(II) chloride (20 ml, in a molar ratio of 1.2:1=sequestering agent: copper(II) chloride, [Cu²⁺]=900 ppm, pH=4.5) was diluted with milliQ-water to a total volume of 60 ml in a beaker, corresponding to a solution with an initial sequestrent- and copper concentration of 5.66 mM and 300 ppm, respectively. To this solution 2 M aqueous hydrogen chloride was added under magnetical stirring at room temperature to pH=1.8. A light blue precipitate was removed by filtration (P3 glass filter) leaving a colourless transparent filtrate. The light blue precipitate was dried in a vacuum chamber at 0.8 mbar for 19 hours resulting in a light blue solid.

### Results

270 mg precipitate was obtained. Organic analyses were made with the aid of an ESI-MS (recorded on a Micromass Quattro II mass spectrometer coupled with a Harvard Apparatus Pump 11 syringe pump directly into the ESI source of the mass spectrometer at a flow rate of 6 μl/min. The data was processed using MassLynx 4.0 software) in positive and negative mode.

The precipitate contained exclusively of 4-dodecyl-3,6,9-tri(carboxymethyl)-3,6,9-triazaundecane diacid and complexed sequestering agent. The filtrate showed no content of 4-dodecyl-3,6,9-tri(carboxymethyl)-3,6,9-triazaundecane diacid. Metal analyses were made with the aid of a Perkin-Elmer AA300 atomic absorption spectrometer. 99% of the copper was found in the precipitate, where the copper was bonded to the sequestering agent and 1% of the copper was found in the filtrate.

Thus, this example showed that 4-dodecyl-3,6,9-tri(carboxymethyl)-3,6,9-triazaundecane diacid complexed with copper could be almost completely removed from the solution.

### Experimental Example 6

### Precipitation/recovery of 2-dodecyl-3-carboxymethyl-3-azapentane diacid from a copper(II) sulfate solution

### Materials and Methods

The recovery of 2-dodecyl-3-carboxymethyl-3-azapentane diacid was performed in a two-step procedure.

In the first stage metal ions was removed from the solution by electrolysis at a specified pH and current intensity: The equipment to perform the electrolysis consisted of a Manson EP-601 rectifier and two platinum electrodes in form of a spring (anode) and a basket (cathode).

To a solution of 2-dodecyl-3-carboxymethyl-3-azapentane diacid and copper in the form of copper(II) sulfate (30 ml, in a molar ratio of 1.2:1=sequestering agent: copper(II) sulfate, [sequestrent]=10.5 mM, [Cu²⁺]=655 ppm) in a beaker was sodium sulphate (corresponding to 50 mg/l) added, in order to receive wished current intensity. Solutions of 1 M aqueous sodium hydroxide was added to adjust the pH to 12. The total volume of the solution was 150 ml. The electrolysis was performed during 50 min. at a current intensity of 300-350 mA.

In the second step the electrolysis solution was acidified with 1 M aqueous hydrogen chloride to pH=2.4. The precipitated material was removed by filtration (glass filter—Schott u.Gen Mainz 1G2) and dried in a vacuum chamber at 0.8 mbar for 24 hours.

### Results

99.6 mg precipitate was obtained. Organic analyses were made with the aid of an ESI-MS (recorded on a Micromass Quattro II mass spectrometer coupled with a Harvard Apparatus Pump 11 syringe pump directly into the ESI source of the mass spectrometer at a flow rate of 6 μl/min. The data was processed using MassLynx 4.0 software) in positive and negative mode, and NMR with a Varian 500 instrument. The precipitate contained exclusively of 2-dodecyl-3-carboxymethyl-3-azapentane diacid.

Metal analyses were made with the aid of a Perkin-Elmer AA300 atomic absorption spectrometer.

0.5% of the copper was found in the precipitate, where the copper was bonded to the sequestering agent, 0.1% of the copper was found in the filtrate and 99.4% of the copper was found on the cathode.

The recovery level of the sequestering agent was 66%.

Thus, this example showed that 2-dodecyl-3-carboxymethyl-3-azapentane diacid complexed with copper could be almost completely removed from the solution separately, according to the invention.

### Experimental Example 7

### Precipitation/recovery of 4-dodecyl-3,6,9-tri(carboxymethyl)-3,6,9-triazaundecane diacid from a copper(II) chloride solution

### Materials and Methods

The recovery of 4-dodecyl-3,6,9-tri(carboxymethyl)-3,6,9-triazaundecane diacid was performed in a two-step procedure.

In the first stage metal ions was removed from the solution by electrolysis at a specified pH and current intensity. The equipment to perform the electrolysis consisted of a Manson EP-60.1 rectifier and two platinum electrodes in form of a spring (anode) and a basket (cathode). The electrodes were separated with a cationic exchange membrane (CMI-7000 Cation exchange membranes—Membranes International INC.)

To a solution of 4-dodecyl-3,6,9-tri(carboxymethyl)-3,6,9-triazaundecane diacid and copper in the form of copper(II) chloride (30 ml, in a molar ratio of 1.2:1=sequestering agent: copper(II) chloride, [Cu²⁺]=900 ppm, pH=4.5) in a beaker was sodium sulphate (corresponding to 0.1 M), in order to receive wished current intensity. The pH of the starting solution was 4.1. The total volume of the solution was 300 ml. The electrolysis was performed during 60 min. at a current intensity of 250-300 mA.

In the second step the electrolysis solution was acidified with 1 M aqueous hydrogen chloride to pH=2.4. The precipitated material was removed by filtration (glass filter—Schott u.Gen Mainz 1G2) and dried in a vacuum chamber at 0.8 mbar for 22 hours.

### Results

198.7 mg precipitate was obtained. Organic analyses were made with the aid of an ESI-MS (recorded on a Micromass Quattro II mass spectrometer coupled with a Harvard Apparatus Pump 11 syringe pump directly into the ESI source of the mass spectrometer at a flow rate of 6 μL/min. The data was processed using MassLynx 4.0 software) in positive and negative mode, and NMR with a Varian 500 instrument. The precipitate contained exclusively of 4-dodecyl-3,6,9-tri(carboxymethyl)-3,6,9-triazaundecane diacid. Metal analyses were made with the aid of a Perkin-Elmer AA300 atomic absorption spectrometer.

0.1% of the copper was found in the precipitate, where the copper was bonded to the sequestering agent, 0.1% of the copper was found in the filtrate and 99.8% of the copper was found on the cathode. The recovery level of the sequestering agent was 69%.

Thus, this example showed that 4-dodecyl-3,6,9-tri(carboxymethyl)-3,6,9-triazaundecane diacid complexed with copper could be almost completely removed from the solution separately, according to the invention.

1-49. (canceled)

50. A method for decreasing the amount of at least one metal ion in a liquid material and in a porous solid material surrounded by a liquid, comprising the steps of:
a) contacting said liquid material or porous solid material surrounded by a liquid, with at least one sequestering agent such that said sequestering agent forms at least one complex with said at least one metal ion;
b) removing said complex from said liquid material; and
c) recovering said sequestering agent and/or said metal ion from said complex wherein step c) comprises
c2) adjusting the pH to about 6-12 by addition of an electrolyte solution;
c3) applying a direct voltage current with a cathode and an anode to said electrolyte solution, whereby said at least one metal ion precipitates as a solid on said cathode by electrochemical reduction; and
c4) removal of said cathode comprising the precipitated, solid metal ions; followed by precipitating the remaining sequestering agent in the solution by adjusting the pH to about 0-7 to obtain an electro neutral solution comprising said sequestering agent in precipitated form; followed by filtration of the formed precipitate.

51. The method according to claim 50, wherein said liquid material or porous solid material surrounded by a liquid, is selected from the group consisting of an aqueous liquid, a soil, a liquid comprising sediments or sludge, a slurry and a leachate.

52. The method according to claim 50, wherein step b) comprises flotation of said complex to provide a foam on top of said liquid material, said foam comprising said complex, and removal of said foam from said liquid material.

53. The method according to claim 50, wherein step b) comprises
b1) precipitating said removed complex by adjusting the pH to about 0-7 to obtain an electro neutral solution comprising said complex of said at least one metal ion and said sequestering agent in precipitated form; followed by filtration of the formed precipitate.

54. The method according to claim 50, wherein said at least one metal ion represents an at least bivalent ion selected from the group consisting of manganese, copper, iron, barium, strontium, calcium, magnesium, beryllium, chromium, ruthenium, iridium, tantalum, cobalt, nickel, zinc, cadmium, mercury, aluminum, lead, titanium, uranium, gadolinium, platina, gold and silver ions.

55. The method according to claim 50, wherein said sequestering agent is represented by formula (I) 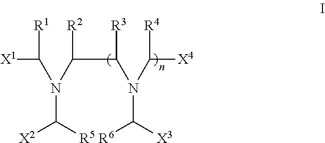
wherein
each of R¹, R², R³, R⁴, R⁵ and R⁶ independently is selected from hydrogen and a straight or branched, saturated or unsaturated hydrocarbon chain having from 9 to 20 carbon atoms, and optionally one or two heteroatoms;
n represents zero, 1 or 3;
X¹, X², X³ and X⁴ is independently selected from the group consisting of hydrogen, —CO₂H, —PO₃H₂, —SO₃H, CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷;
R⁷ represents a straight or branched, saturated or unsaturated hydrocarbon chain having from 9 to 20 carbon atoms, wherein 1 or 2 carbon atoms are optionally substituted with one or two heteroatoms;
provided that at least one of R¹, R², R³, R⁴, R⁵ and R⁶ represents said hydrocarbon chain; or if R¹, R², R³, R⁴, R⁵ and R⁶ represents hydrogen, at least one of X¹, X², X³ and X⁴ represents CO₂R⁷, —CONHR⁷, —CH₂OR⁷, —COR⁷, —CH₂OCOR⁷, —CH₂OCONHR⁷, —PO₃HR⁷, —PO₃(R⁷)₂ or —SO₃R⁷; and salts, stereoisomers and mixtures thereof.

56. The method according to claim 55, wherein n is zero, and X¹ and X² are independently selected from the group consisting of —CO₂H, —PO₃H₂ and —SO₃H.

57. The method according to claim 55, wherein n is 1, and X¹, X², X³ and X⁴ are independently selected from —CO₂H, —PO₃H₂ and —SO₃H.

58. The method according to claim 55, wherein at least one of R¹, R², R³, R⁴, R⁵ and R⁶ represents a straight hydrocarbon chain having 12 carbon atoms.

59. The method according to claim 55, wherein R¹, R², R³, R⁴, R⁵ and R⁶ represents hydrogen; at least one of X¹, X², X³ and X⁴ is independently selected from CO₂R⁷, —CONHR⁷, —CH₂OR⁷, —COR⁷, —CH₂OCOR⁷, —CH₂OCONHR⁷, —PO₃HR⁷, —PO₃(R⁷)₂ and —SO₃R⁷; and
the remaining X¹, X², X³ and X⁴ is independently selected from —CO₂H, —PO₃H₂, and —SO₃H.

60. The method according to claim 59, wherein R⁷ represents a straight hydrocarbon chain having 12 carbon atoms.

61. The method according to claim 56, wherein said agent is selected from

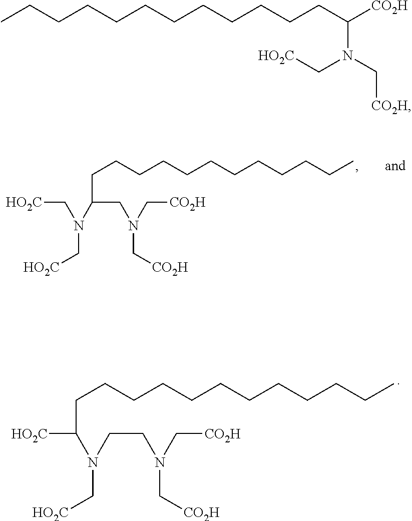

62. A sequestering agent represented by formula (I) 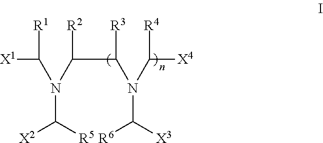
wherein
each of R¹, R², R³, R⁴, R⁵ and R⁶ independently is selected from hydrogen and a straight or branched, saturated or unsaturated hydrocarbon chain having from 9 to 20 carbon atoms, and optionally one or two heteroatoms, provided that at least one of R¹, R², R³, R⁴, R⁵ and R⁶ represents said hydrocarbon chain;
n represents zero, 1 or 3;
X¹, X², X³ and X⁴ is independently selected from hydrogen, —CO₂H, —PO₃H₂, —SO₃H, CO₂R⁷, —CONHR⁷, —CH₂OR⁷, —COR⁷, —CH₂OCOR⁷,
—CH₂OCONHR⁷, —PO₃HR⁷, —PO₃(R⁷)² and —SO₃R⁷;
R⁷ represents a straight or branched, saturated or unsaturated hydrocarbon chain having from 9 to 20 carbon atoms, wherein 1 or 2 carbon atoms are optionally substituted with one or two heteroatoms;
provided that when n is zero; X¹, X² and X⁴ is selected from —PO₃H₂ and —SO₃H; and
provided that when n is 1, at least three of X¹, X², X³ and X⁴ is selected from —CO₂H, —PO₃H₂ and —SO₃H; and
provided that when n is 1; X¹, X², X³ and X⁴ represents —CO₂H; R², R³, R⁴, R⁵ and R⁶ represents hydrogen; then R¹ is not a straight hydrocarbon chain having 10 or 14 carbon atoms; and
provided that when n is 1; X¹, X², X³ and X⁴ represents —CO₂H; R¹, R³, R⁴, R⁵ and R⁶ represents hydrogen; then R² is not a straight hydrocarbon chain having 10, 12 or 14 carbon atoms; and
provided that when n is 1; X¹, X², X³ and X⁴ represents —CO₂H; R², R³, R⁵ and R⁶ represents hydrogen; then R¹ and R⁴ is not a straight hydrocarbon chain having 10 or 12 carbon atoms; and R¹ is not a straight hydrocarbon chain having 10 carbon atoms and R⁴ is not a straight hydrocarbon chain having 12 carbon atoms at the same time; and
provided that when n is 1; X¹, X², X³ and X⁴ represents —CO₂H; R¹, R⁴, R⁵ and R⁶ represents hydrogen; then R² and R³ is not a straight hydrocarbon chain having 10 or 12 carbon atoms; and R² is not a straight hydrocarbon chain having 10 carbon atoms and R³ is not a straight hydrocarbon chain having 12 carbon atoms at the same time; and
provided that when n is 1; X², X³ and X⁴ represents —CO₂H; and X¹ represents CH₂CONR⁷, then R⁷ is not a straight hydrocarbon chain having 10, 12 or 14 carbon atoms; and
provided that when n is 1; X², X³ and X⁴ represents —CO₂H; and X¹ represents CH₂CO₂R⁷, then R⁷ is not a straight hydrocarbon chain having 10, 12, 14, 16 or 18 carbon atoms; or when X¹ represents CH₂OCOR⁷, then R⁷ is not a straight hydrocarbon chain having 17 carbon atoms; and salts, stereoisomers and mixtures thereof.

63. A sequestering agent according to claim 62, said agent being selected from

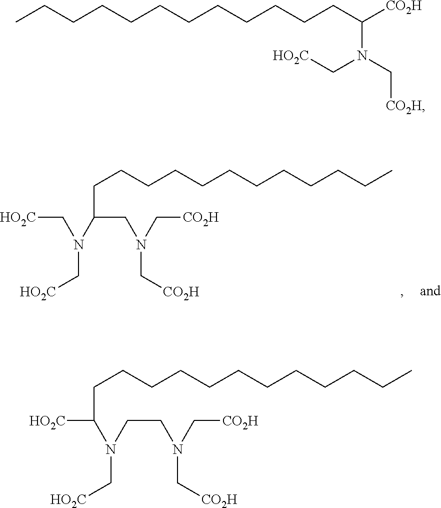

64. A composition comprising at least one sequestering agent according to claim 62.
